(19) United States
(12) Patent Application Publication
(10) Pub. No.: US 2026/0086096 A1
(43) Pub. Date: Mar. 26, 2026

(54) ENDOGLIN ANTIBODIES AND USES THEREOF

(71) Applicant: ARIZONA BOARD OF REGENTS ON BEHALF OF THE UNIVERSITY OF ARIZONA, Tucson, AZ (US)

(72) Inventor: Nam Yong Lee, Tucson, AZ (US)

(21) Appl. No.: 19/341,471

(22) Filed: Sep. 26, 2025

Related U.S. Application Data
(60) Provisional application No. 63/699,473, filed on Sep. 26, 2024.

(51) Int. Cl. G01N 33/574; C07K 16/28; G01N 33/543

(52) U.S. Cl. CPC G01N 33/5759; C07K 16/2896; G01N 33/543; G01N 33/57515; G01N 33/57525; G01N 33/57535; C07K 2317/14; G01N 2333/70596

(57) ABSTRACT

This disclosure provides methods and compositions for detecting splice variants of the endoglin protein, enabling the accurate diagnosis of cancer. Additionally, the use of endoglin-specific antibodies enhances the detection process, offering a more precise and reliable approach to identifying cancer-related variants of endoglin.

### CROSS-REFERENCE TO RELATED APPLICATIONS

This application is a non-provisional and claims benefit of U.S. Provisional Application No. 63/699,473 filed Sep. 26, 2024, the specification of which is incorporated herein in its entirety by reference.

### REFERENCE TO AN ELECTRONIC SEQUENCE LISTING

The contents of the electronic sequence listing (ARIZ_24_32_NP_Sequence_Listing.xml; Size: 6,833 bytes; and Date of Creation: Dec. 4, 2025) is herein incorporated by reference in its entirety.

### FIELD OF THE INVENTION

The present invention provides methods and compositions for detecting splice variants of the endoglin protein, enabling the accurate diagnosis of cancer. This invention further includes the use of endoglin-specific antibodies to enhance the detection process, offering a more precise and reliable approach to identifying cancer-related variants of endoglin.

### BACKGROUND OF THE INVENTION

Pancreatic cancer is a lethal malignancy characterized by early metastasis, late-onset symptoms, and a notorious resistance to existing therapies. A critically elusive aspect of this disease involves the tumors, which are often hypovascularized compared to other solid cancers, resulting in poor perfusion and impaired drug delivery.

The present invention surprisingly found that endoglin, an endothelial-specific TGF-beta coreceptor essential for angiogenesis, is expressed in pancreatic cancer cells as two variants: the wildtype, which supports tumor-intrinsic growth and chemoresistance, and a novel splice variant with distinct structural features that is secreted to inhibit tumor vascularization, as described herein. Thus, the present invention features anti-endoglin antibodies that specifically target the novel splice variants of the endoglin protein. The use of the aforementioned endoglin-specific antibodies enhances the detection process, offering a more precise and reliable approach to identifying cancer-related variants of endoglin.

### BRIEF SUMMARY OF THE INVENTION

It is an objective of the present invention to provide systems, compositions, and methods that allow for the detection of splice variants of the endoglin protein, enabling the accurate diagnosis of cancer, as specified in the independent claims. Embodiments of the invention are given in the dependent claims. Embodiments of the present invention can be freely combined with each other if they are not mutually exclusive.

In some embodiments, the present invention features a means for binding to an endoglin splice variant (e.g., an anti-endoglin antibody). In some embodiments, the endoglin splice variant is according to SEQ ID NO: 2 or SEQ ID NO: 3. In some embodiments, the endoglin splice variant is at least 75%, 80%, 85%, 90%, 95%, or 98% identical to SEQ ID NO: 2 or SEQ ID NO: 3.

In some embodiments, the present invention features a method of generating an anti-endoglin antibody, the method comprising injecting a synthetic peptide according to either SEQ ID NO: 4 or SEQ ID NO: 5 into a host animal and recovering the anti-endoglin antibodies from the host animal. In some embodiments, the synthetic peptide elicits an immune response and generates anti-endoglin antibodies. In some embodiments, the synthetic peptide is at least 75%, 80%, 85%, 90%, 95%, or 98% identical to SEQ ID NO: 4 or SEQ ID NO: 5. In some embodiments, the synthetic peptide is conjugated to a carrier. In some embodiments, the synthetic peptide is conjugated to bovine serum albumin (BSA) or albumin.

In some embodiments, the present invention features a method of detecting an endoglin splice variant in a subject, the method comprising obtaining or having obtained a biological sample from the subject, detecting or having detected an endoglin splice variant in the biological sample by contacting the biological sample with an anti-endoglin antibody, and detecting binding between the endoglin splice variant and the antibody. In some embodiments, the biological sample is a liquid biopsy. In some embodiments, the liquid biopsy is a blood sample. In some embodiments, the subject has pancreatic cancer, colon cancer, breast cancer, another cancer, or another malignancy. In some embodiments, the splice variants are according to SEQ ID NO: 2 or SEQ ID NO: 3. In some embodiments, the splice variant is at least 75%, 80%, 85%, 90%, 95%, or 98% identical to SEQ ID NO: 2 or SEQ ID NO: 3.

In some embodiments, the splice variants of endoglin are detected by sequencing. In some embodiments, the splice variants of endoglin are detected by an immunoassay. In some embodiments, the immunoassay is an enzyme-linked immunosorbent assay (ELISA).

In some embodiments, the ELISA comprises a substrate and a means for binding to an endoglin splice variant (i.e., an antigen), the endolin splice variant according to SEQ ID NO: 2 or SEQ ID NO: 3. In some embodiments, the endoglin splice variant is at least 75%, 80%, 85%, 90%, 95%, or 98% identical to SEQ ID NO: 2 or SEQ ID NO: 3. In some embodiments, the means for binding to the endoglin splice variant is an anti-endoglin antibody configured to bind to an epitope of the antigen, the epitope according to SEQ ID NO: 4 or SEQ ID NO: 5.

In some embodiments, the present invention features an anti-endoglin antibody configured to bind an endoglin splice variant, the antibody specifically binding to an epitope, the epitope being encoded by SEQ ID NO: 4 or SEQ ID NO: 5, the antibody obtained by a process comprising injecting a synthetic peptide that is at least 75% identical to SEQ ID NO: 4 or SEQ ID NO: 5 into a host animal and recovering the anti-endoglin antibodies produced thereby from the host animal. In some embodiments, the synthetic peptide is at least 80%, 85%, 90%, 95%, or 98% identical to SEQ ID NO: 4 or SEQ ID NO: 5.

One of the unique and inventive technical features of the present invention is anti-endoglin antibodies that specifically target splice variants of the endoglin protein. Without wishing to limit the invention to any theory or mechanism, it is believed that the technical feature of the present invention advantageously provides for specific diagnosis, prognosis, and treatment of cancer, e.g., pancreatic cancer (see FIG. 2A-2B). None of the presently known prior references or works have the unique inventive technical feature of the present invention.

Furthermore, the inventive technical feature of the present invention led to a surprising discovery: the identification of two splice variants of the endoglin protein that are exclusively associated with cancer. This finding enabled the development of specific antibodies designed to target these unique splice variants.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows the wild-type version of an endoglin protein.

FIG. 1B shows two splice variants of the endoglin protein. The splice variant pictured at the top, having 352 amino acids, is splice variant Eng2 (SEQ ID NO: 3). The splice variant pictured at the bottom, having 404 amino acids, is splice variant Eng1 (SEQ ID NO: 2).

FIGS. 2A-2B show the detection of endoglin splice variant 1 (Eng1) in human pancreatic cancer. Collectively, FIGS. 2A-2B show that the endoglin splice variant Eng1 is expressed in human pancreatic cancer but not normal pancreatic tissue, that the antibodies of the present invention selectively detect Eng1 via immunohistochemical (IHC), and that Eng1 and Eng2 may serve as diagnostic and/or prognostic markers of pancreatic cancer. FIG. 2A shows IHC detection of endoglin splice variant 1 (Eng1) in human pancreatic cancer tissue. Representative IHC images are shown from a tissue microarray containing 70 pancreatic cancer patient samples across various clinical stages. Samples were stained with an antibody specific to the intron retention-encoded peptide sequence WTSGSSPSPRLL (SEQ ID NO: 4). FIG. 2B shows quantification of Eng1 staining in normal pancreatic tissue versus tumor samples stratified by clinical stage (I-IV).

### DETAILED DESCRIPTION OF THE INVENTION

Unless otherwise explained, all technical and scientific terms used herein have the same meaning as commonly understood by one of ordinary skill in the art to which a disclosed invention belongs. All publications, patent applications, patents, and other references mentioned herein are incorporated by reference in their entirety for all purposes. In case of conflict, the present specification, including explanations of terms, will control.

The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessarily solely”. Furthermore, variations of the word “comprising,” such as “comprise” and “comprises”, have correspondingly the same meanings. In one respect, the technology described herein is related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

Although methods and materials similar or equivalent to those described herein can be used to practice or test the disclosed technology, suitable methods and materials are described below. The materials, methods, and examples are illustrative only and not intended to be limiting.

As used herein, the terms “subject” and “patient” are used interchangeably. As used herein, a subject can be a mammal such as a non-primate (e.g., cows, pigs, horses, cats, dogs, rats, etc.) or a primate (e.g., monkey and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human.

“Signal producing component” refers to any substance capable of reacting with another assay reagent or with the biomarker(s) to produce a reaction product or signal that indicates the presence of the biomarker(s) and that is detectable by visual or instrumental means. “Signal production system,” as used herein, refers to the group of assay reagents that are needed to produce the desired reaction product or signal.

“Observable signal,” as used herein, refers to a signal produced in the claimed devices and methods that is detectable by visual inspection, and/or by ELISA-based fluorescence and/or colorimetric assays. Generally, observable signals indicating the presence or absence of a biomarker in a sample may be evident of their own accord, e.g., plus or minus signs or particularly shaped symbols, or may be evident through the comparison with a panel, such as a color indicator panel.

As used herein, the terms “upstream” and “downstream” refer to the direction of a sample flow subsequent to contact of the sample with a representative device of the present disclosure, wherein, under normal operating conditions, the fluid sample flow direction runs from an upstream position to a downstream position. For example, when a fluid sample is initially contacted with the sample receiving zone, the fluid sample then flows downstream through the conjugating zone and so forth.

As used herein, the terms “treat,” “treating,” or “treatment” refer to both therapeutic treatment and prophylactic or preventative measures, with the objective of preventing, reducing, slowing down (lessen), inhibiting, or eliminating an undesired physiological change, symptom, disease, or disorder. For example, the disease may be cancer, e.g., pancreatic cancer, breast cancer, colon cancer, prostate cancer, and/or the like. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented or onset delayed. Optionally, the subject or patient may be identified (e.g., diagnosed) as one suffering from the disease or condition prior to administration of the compositions of the invention. Subjects at risk for the disease can be identified by, for example, any or a combination of appropriate diagnostic or prognostic assays known in the art.

The terms “polypeptide” and “protein” are used interchangeably to refer to a polymer of amino acid residues, comprising natural or non-natural amino acid residues, and are not limited to a minimum length. Thus, peptides, oligopeptides, dimers, multimers, and the like are included within the definition. Both full-length proteins and fragments thereof are encompassed by the definition. The terms also include post-translational modifications of the polypeptide, including, for example, glycosylation, sialylation, acetylation, and phosphorylation. Furthermore, a “polypeptide” herein also refers to a modified protein, such as single or multiple amino acid residue deletions, additions, and substitutions to the native sequence, as long as the protein maintains a desired activity. For example, a serine residue may be substituted to eliminate a single reactive cysteine or to remove disulfide bonding or a conservative amino acid substitution may be made to eliminate a cleavage site. These modifications may be deliberate, as through site-directed mutagenesis, or may be accidental, such as through mutations of hosts, which produce the proteins or errors due to polymerase chain reaction (PCR) amplification.

As used herein, the term “peptide” refers to a short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein or a non-natural (synthetic) sequence.

As used herein, the term “wildtype” refers to a non-mutated version of a gene, allele, genotype, polypeptide, phenotype, or a fragment of any of these. It may occur in nature or be produced recombinantly. As used herein, the term “variant” refers to a nucleic acid molecule or polypeptide that differs from a referent nucleic acid molecule or polypeptide by single or multiple amino acid substitutions, deletions, and/or additions and substantially retains at least one biological activity of the referent nucleic acid molecule or polypeptide.

As used herein, a “biological sample” or “clinical sample” may be used interchangeably and may refer to any biological material taken from a subject. Non-limiting examples of a biological material to be taken for a biological sample may include, but are not limited to, saliva, blood, or urine, and nasopharyngeal specimens.

As used herein, an “antibody (Ab)” may refer to a polypeptide that includes at least a light chain and/or heavy chain immunoglobulin variable region and specifically binds an epitope of an antigen. Heavy chain antibodies, such as those found in sharks and camelids (the precursors for nanobodies), are also included in this definition. Most commonly, an antibody refers to primary recombinant monoclonal antibodies or mAbs. Antibodies include monoclonal antibodies, polyclonal antibodies, or fragments of antibodies. Merely by way of example, monoclonal antibodies can be prepared from murine hybridomas according to classical methods such as Kohler and Milstein (Nature 256:495-97, 1975) or derivative methods thereof. Detailed procedures for monoclonal antibody production are described, for example, by Harlow and Lane, Using Antibodies: A Laboratory Manual, CSHL, New York, 1999. An antibody can be conjugated or otherwise labeled with a detectable label, such as a fluorophore (e.g., a reporter).

As used herein, an “antibody fragment” refers to a partial or truncated version of an antibody that retains the ability to specifically bind to its target antigen. Non-limiting examples of antibody fragments include but are not limited to Fab, F(ab′)2, Fab′, Fv, scFv, sdAb, VHH, and dAb fragments.

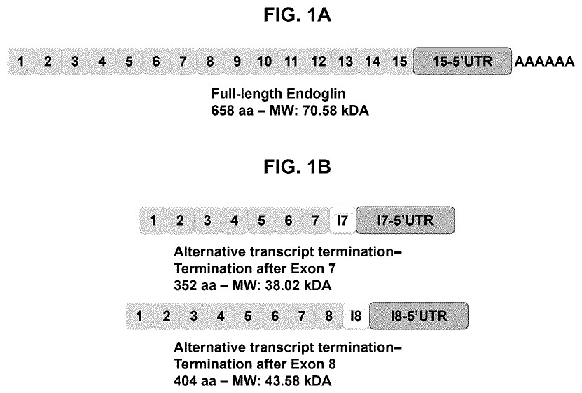

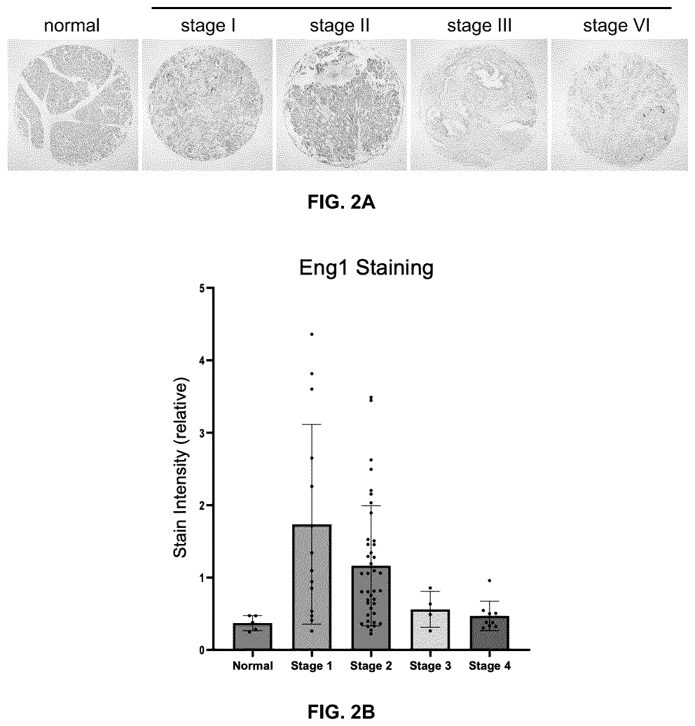

Referring to FIG. 1A-2B, the present invention provides systems, compositions, and methods that allow for the detection of a splice variant of an endoglin protein. Additionally, the present invention may feature systems, compositions, and methods for diagnosing and treating cancer, e.g., pancreatic cancer, in a subject.

Endoglin is expressed mostly in proliferating endothelial cells. It plays an essential role in angiogenesis during development and pathological settings. Below is the canonical protein sequence of endoglin, a single transmembrane cell surface receptor.

| | | SEQ |
|---|---|---|
| | | ID |
| | | NO: |
| Full- | MDRGTLPLAVALLLASCSLSPTSLAETVHCD | 1 |
| length | LQPVGPERGEVTYTTSQVSKGCVAQAPNAIL | |
| canonical | EVHVLFLEFPTGPSQLELTLQASKQNGTWPR | |
| endoglin | EVLLVLSVNSSVFLHLQALGIPLHLAYNSSL | |
| protein | VTFQEPPGVNTTELPSFPKTQILEWAAERGP | |
| sequence | ITSAAELNDPQSILLRLGQAQGSLSFCMLEA | |
| | SQDMGRTLEWRPRTPALVRGCHLEGVAGHKE | |
| | AHILRVLPGHSAGPRTVTVKVELSCAPGDLD | |
| | AVLILQGPPYVSWLIDANHNMQIWTTGEYSF | |
| | KIFPEKNIRGFKLPDTPQGLLGEARMLNASI | |
| | VASFVELPLASIVSLHASSCGGRLQTSPAPI | |
| | QTTPPKDTCSPELLMSLIQTKCADDAMTLVL | |
| | KKELVAHLKCTITGLTFWDPSCEAEDRGDKF | |
| | VLRSAYSSCGMQVSASMISNEAWVNILSSSS | |
| | PQRKKVHCLNMDSLSFQLGLYLSPHFLQASN | |
| | TIEPGQQSFVQVRVSPSVSEFLLQLDSCHLD | |
| | LGPEGGTVELIQGAAKGNCVSLLSPSPEGDP | |
| | RFSFLLHFYTVPIPKTGTLSCTVALRPKTGS | |
| | QDQEVHRTVFMRLNIISPDLSGCTSKGLVLP | |
| | AVLGITFGAFLIGALLTAALWYIYSHTRSPS | |
| | KREPVVAVAAPASSESSSTNHSIGSTQSTPC | |
| | STSSMA | |

However, the endoglin full-length receptor was more recently detected in pancreatic, colon, and breast cancer cells. The Inventors have identified at least two major alternative splice variants expressed in pancreatic cancer cells. These variants were subsequently cloned, and their sequences are provided below. The protein sequences show parts of the WT endoglin extracellular domain followed by short peptides derived from partial read-throughs of either intron 7 or intron 8 (intron retention). Underlined/Italicized regions are the intron-derived peptide sequences that are unusual and correlate specifically with pancreatic cancer but not normal cells. This intron-derived peptide sequence has no homology to known proteins. Bolded regions indicate antigens used to generate antibodies described herein.

| | | SEQ |
|---|---|---|
| | | ID |
| | Sequence | NO: |
| Eng1 | MDRGTLPLAVALLLASCSLSP | 2 |
| | TSLAETVHCDLQPVGPERGEV | |
| | TYTTSQVSKGCVAQAPNAILE | |
| | VHVLFLEFPTGPSQLELTLQA | |
| | SKQNGTWPREVLLVLSVNSSV | |
| | FLHLQALGIPLHLAYNSSLVT | |
| | FQEPPGVNTTELPSFPKTQIL | |
| | EWAAERGPITSAAELNDPQSI | |
| | LLRLGQAQGSLSFCMLEASQD | |
| | MGRTLEWRPRTPALVRGCHLE | |
| | GVAGHKEAHILRVLPGHSAGP | |
| | RTVTVKVELSCAPGDLDAVLI | |
| | LQGPPYVSWLIDANHNMQIWT | |
| | TGEYSFKIFPEKNIRGFKLPD | |
| | TPQGLLGEARMLNASIVASFV | |
| | ELPLASIVSLHASSCGGRLQT | |
| | SPAPIQTTPPKDTCSPELLMS | |
| | LIQTKCADDAMTLVLKKELVA | |
| | VRELLPLWLRMTWTSGSSPSP | |
| | RLLGS | |
| Eng2 | MDRGTLPLAVALLLASCSLSP | 3 |
| | TSLAETVHCDLQPVGPERGEV | |
| | TYTTSQVSKGCVAQAPNAILE | |
| | VHVLFLEFPTGPSQLELTLQA | |
| | SKQNGTWPREVLLVLSVNSSV | |
| | FLHLQALGIPLHLAYNSSLVT | |
| | FQEPPGVNTTELPSFPKTQIL | |
| | EWAAERGPITSAAELNDPQSI | |
| | LLRLGQAQGSLSFCMLEASQD | |
| | MGRTLEWRPRTPALVRGCHLE | |
| | GVAGHKEAHILRVLPGHSAGP | |
| | RTVTVKVELSCAPGDLDAVLI | |
| | LQGPPYVSWLIDANHNMQIWT | |
| | TGEYSFKIFPEKNIRGFKLPD | |
| | TPQGLLGEARMLNASIVASFV | |
| | ELPLASIVSLHASSCGEHPSP | |
| | APPFPSPPLDQWPHCW | |
| Anti- | WTSGSSPSPRLL | 4 |
| gen 1 | | |
| Anti- | GEHPSPAPPFPSPP | 5 |
| gen 2 | | |

The present invention may feature a means for binding to an endoglin splice variant (e.g., Eng1 or Eng2). Alternatively, in some embodiments, the present invention may also feature an anti-endoglin antibody configured to bind to an endoglin splice variant. In some embodiments, the endoglin splice variant is according to SEQ ID NO: 2 or SEQ ID NO: 3. In other embodiments, the endoglin splice variant is at least 75%, 80%, 85%, 90%, 95%, or 98% identical to SEQ ID NO: 2 or SEQ ID NO: 3.

In certain embodiments, the means for binding to the endoglin splice variant comprises an anti-endoglin antibody configured to bind to an antigen (e.g., an endoglin splice variant) according to SEQ ID NO: 2 or SEQ ID NO: 3. In some embodiments, the antigen (e.g., splice variant) is at least 75%, 80%, 85%, 90%, 95%, or 98% identical to SEQ ID NO: 2 or SEQ ID NO: 3.

In some embodiments, the present invention features a process for producing an anti-endoglin antibody configured to bind an endoglin splice variant. In some embodiments, the anti-endoglin antibody is produced by a process comprising the steps of: injecting a synthetic peptide that is at least 75% identical to SEQ ID NO: 4 or SEQ ID NO: 5 into a host animal and recovering the anti-endoglin antibodies thereby produced from the host animal. In other embodiments, the anti-endoglin antibody is produced by a process comprising the steps of: injecting a synthetic peptide that is at least 80%, 85%, 90%, 95%, 98%, or 100% identical to SEQ ID NO: 4 or SEQ ID NO: 5 into a host animal and recovering the anti-endoglin antibodies thereby produced from the host animal. In some embodiments, the antibody is configured to specifically bind to an epitope on the endogline splice variant (e.g., antigen). In some embodiments, the antigen is encoded by SEQ ID NO: 2 or SEQ ID NO: 3.

The present invention may also feature a method of generating an anti-endoglin antibody, e.g., an anti-endoglin antibody configured to bind to an endoglin splice variant. In some embodiments, the method comprises a) injecting a synthetic peptide according to either SEQ ID NO: 4 or SEQ ID NO: 5 into a host animal and b) recovering the anti-endoglin antibodies from the host animal. In other embodiments, the method comprises a) injecting a synthetic peptide that is at least 75%, 80%, 85%, 90%, 95%, or 98% identical to SEQ ID NO: 4 or SEQ ID NO: 5 into a host animal and b) recovering the anti-endoglin antibodies from the host animal. In some embodiments, the synthetic peptide elicits an immune response and generates anti-endoglin antibodies.

In some embodiments, the synthetic peptides are according to SEQ ID NO: 4 or SEQ ID NO: 5. In some embodiments, the synthetic peptide is at least 75% identical to SEQ ID NO: 4 or SEQ ID NO: 5. In some embodiments, the synthetic peptide is at least 80% identical to SEQ ID NO: 4 or SEQ ID NO: 5. In some embodiments, the synthetic peptide is at least 85% identical to SEQ ID NO: 4 or SEQ ID NO: 5. In some embodiments, the synthetic peptide is at least 90% identical to SEQ ID NO: 4 or SEQ ID NO: 5. In some embodiments, the synthetic peptide is at least 95% identical to SEQ ID NO: 4 or SEQ ID NO: 5. In some embodiments, the synthetic peptide is at least 98% identical to SEQ ID NO: 4 or SEQ ID NO: 5. In some embodiments, the synthetic peptide is at least 99% identical to SEQ ID NO: 4 or SEQ ID NO: 5. In some embodiments, the synthetic peptide is at least 100% identical to SEQ ID NO: 4 or SEQ ID NO: 5.

In some embodiments, the synthetic peptide is conjugated to a carrier, e.g., BSA or albumin. In some embodiments, the synthetic peptide is conjugated to bovine serum albumin (BSA) or albumin. In some embodiments, the host animal is a mouse, rat, or rabbit.

Additionally, the present invention may feature a method of detecting an endoglin splice variant in a subject. In some embodiments, the method comprises obtaining or having obtained a biological sample from the subject, detecting or having detected an endoglin splice variant in the biological sample by contacting the biological sample with an anti-endoglin antibody, and detecting binding between the endoglin splice variant and the antibody.

Additionally, the present invention may feature a method of diagnosing cancer (e.g., pancreatic cancer) in a subject in need thereof. In some embodiments, the method comprises a) obtaining or having obtained a biological sample from the subject, b) detecting or having detected an endoglin protein in the biological sample, and c) diagnosing cancer in the subject if a splice-variant of the endoglin protein is detected. In some embodiments, detecting the splice variant comprises detecting binding between the endoglin splice variant and an antibody or a means for binding an endoglin splice variant, as described herein.

In some embodiments, the biological sample is a liquid biopsy. In some embodiments, the liquid biopsy is a blood sample. In some embodiments, the biological sample is a solid biopsy. In some embodiments, the solid biopsy is a solid tumor biopsy. In some embodiments, the cancer is a hypovascularized cancer. In some embodiments, the cancer comprises a solid tumor. In some embodiments, the cancer is pancreatic, colon, or breast cancer.

In some embodiments, the splice variants are according to SEQ ID NO: 2 or SEQ ID NO: 3. In some embodiments, the splice variant is at least 75% identical to SEQ ID NO: 2 or SEQ ID NO: 3. In some embodiments, the splice variant is at least 80% identical to SEQ ID NO: 2 or SEQ ID NO: 3. In some embodiments, the splice variant is at least 85% identical to SEQ ID NO: 2 or SEQ ID NO: 3. In some embodiments, the splice variant is at least 90% identical to SEQ ID NO: 2 or SEQ ID NO: 3. In some embodiments, the splice variant is at least 95% identical to SEQ ID NO: 2 or SEQ ID NO: 3. In some embodiments, the splice variant is at least 98% identical to SEQ ID NO: 2 or SEQ ID NO: 3. In some embodiments, the splice variant is at least 99% identical to SEQ ID NO: 2 or SEQ ID NO: 3. In some embodiments, the splice variant is at least 100% identical to SEQ ID NO: 2 or SEQ ID NO: 3.

In some embodiments, the splice variants of endoglin are detected by sequencing. In other embodiments, the splice-variants of endoglin are detected by an immunoassay, e.g., an enzyme-linked immunosorbent assay (ELISA).

In some embodiments, the immunoassay comprises a substrate and an antibody or a means for binding an endoglin splice variant, as described herein, conjugated to the substrate. For example, the ELISA may comprise a substrate and an antibody or a means for binding an endoglin splice variant, as described herein, conjugated to the substrate. The antibody conjugated to the substrate is configured to bind (e.g., capture) to an endoglin splice variant. In some embodiments, the endoglin splice variant is according to SEQ ID NO: 2 or SEQ ID NO: 3. In other embodiments, the splice variant is at least 75%, 80%, 85%, 90%, 95%, or 98% identical to SEQ ID NO: 2 or SEQ ID NO: 3.

The present invention may further feature a method for detecting the presence of a splice variant of an endoglin protein in a biological sample. In some embodiments, the method comprises a) contacting a biological sample to a substrate comprising a first antibody, e.g., an anti-endoglin antibody configured to bind to an endoglin splice variant, conjugated thereto, b) contacting the substrate with a second antibody, wherein the second antibody is conjugated to a reporter, and c) detecting the signal. In some embodiments, when the splice variant of the endoglin protein binds to both the first antibody and the second antibody, a signal is produced by the reporter. In some embodiments, if a signal is detected, a biopsy or scan is performed.

Alternatively, the present invention may further feature a method for detecting the presence of an anti-endoglin antibody in a biological sample. In some embodiments, the method comprises a) contacting a biological sample to a substrate comprising an antigen, e.g., an endoglin splice variant, conjugated thereto, b) contacting the substrate with a secondary antibody, wherein the secondary antibody is conjugated to a reporter, and c) detecting the signal. In some embodiments, when the anti-endoglin antibody binds to both the antigen and the secondary antibody, a signal is produced by the reporter. In some embodiments, if a signal is detected, a biopsy or scan is performed.

In some embodiments, the present invention features a composition comprising an anti-endoglin antibody or a means for binding to the endoglin splice variant, as described herein, conjugated to a therapeutic agent.

Referring to FIGS. 2A-2B specifically, the results depicted in FIGS. 2A-2B demonstrate that the endoglin splice variant Eng1 is expressed in human pancreatic cancer but not normal pancreatic tissue, the antibodies of the present invention selectively detect Eng1 via IHC, and that Eng1 and Eng2 may serve as diagnostic/prognostic marker(s) of pancreatic cancer. Furthermore, because Eng1 and Eng2 both lack the transmembrane domain sequence of the full-length wildtype endoglin, Eng1 and/or Eng2 may be readily secreted into the tumor microenvironment and/or into circulation, thus enabling potential detection by non-invasive, blood-based screening methods such as ELISA using antibodies of the present invention directed against Antigen 1 and/or Antigen 2.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A means for binding to an endoglin splice variant, wherein the endoglin splice variant is according to SEQ ID NO: 2 or SEQ ID NO: 3.

2. The means of claim 1, wherein the endoglin splice variant is at least 75%, 80%, 85%, 90%, 95%, or 98% identical to SEQ ID NO: 2 or SEQ ID NO: 3.

3. A method of generating a means for binding to an endoglin splice variant according to claim 1, the method comprising:
a) injecting a synthetic peptide according to either SEQ ID NO: 4 or SEQ ID NO: 5 into a host animal;
wherein the synthetic peptide elicits an immune response and generates the means for binding to an endoglin splice variant; and
b) recovering the means for binding to an endoglin splice variant from the host animal.

4. The method of claim 3, wherein the synthetic peptide is at least 75%, 80%, 85%, 90%, 95%, or 98% identical to SEQ ID NO: 4 or SEQ ID NO: 5.

5. The method of claim 3, wherein the synthetic peptide is conjugated to a carrier.

6. The method of claim 5, wherein the carrier is bovine serum albumin or albumin; wherein the synthetic peptide is conjugated to bovine serum albumin (BSA) or albumin.

7. A method of detecting an endoglin splice variant in a subject, the method comprising:
a) obtaining or having obtained a biological sample from the subject;
b) detecting or having detected an endoglin splice variant in the biological sample by contacting the biological sample with an anti-endoglin antibody and detecting binding between the endoglin splice variant and the antibody.

8. The method of claim 7, wherein the biological sample is a liquid biopsy.

9. The method of claim 8, wherein the liquid biopsy is a blood sample.

10. The method of claim 7, wherein the subject has pancreatic cancer, colon cancer, or breast cancer.

11. The method of claim 7, wherein the endoglin splice variant comprises a sequence according to either SEQ ID NO: 2 or SEQ ID NO: 3.

12. The method of claim 7, wherein the endoglin splice variant is at least 75%, 80%, 85%, 90%, 95%, or 98% identical to SEQ ID NO: 2 or SEQ ID NO: 3.

13. The method of claim 7, wherein the endoglin splice-variant is detected by sequencing.

14. The method of claim 7, wherein the endoglin splice-variant is detected by an immunoassay.

15. The method of claim 14, wherein the immunoassay is an enzyme-linked immunosorbent assay (ELISA).

16. The method of claim 15, wherein the ELISA comprises:
a) a substrate; and
b) a means for binding to an endoglin splice variant according to SEQ ID NO: 2 or SEQ ID NO: 3.

17. The method of claim 16, wherein the means for binding to the endoglin splice variant is an anti-endoglin antibody configured to bind to an epitope of the endoglin splice variant comprising a sequence according to either SEQ ID NO: 4 or SEQ ID NO: 5.

18. The method of claim 16, wherein the splice variant is at least 75%, 80%, 85%, 90%, 95%, or 98% identical to SEQ ID NO: 2 or SEQ ID NO: 3.

19. An anti-endoglin antibody configured to bind an endoglin splice variant the antibody obtained by a process comprising:
a) injecting a synthetic peptide that is at least 75% identical to SEQ ID NO: 4 or SEQ ID NO: 5 into a host animal;
wherein the synthetic peptide elicits an immune response and generates the anti-endoglin antibodies, and
b) recovering the anti-endoglin antibodies produced in step a) from the host animal.

20. The anti-endoglin antibody of claim 18, wherein the anti-endoglin antibody is at least 80%, 85%, 90%, 95%, or 98% identical to SEQ ID NO: 4 or SEQ ID NO: 5.


SEQUENCE LISTING (5 sequences)

SEQ ID NO: 1 - protein, 657 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 2 - protein, 404 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAVRELLPLWLRMTWTSGSSPSPRLLGS

SEQ ID NO: 3 - protein, 352 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGEHPSPAPPFPSPPLDQWPHCW

SEQ ID NO: 4 - protein, 12 aa, Homo sapiens
WTSGSSPSPRLL

SEQ ID NO: 5 - protein, 14 aa, Homo sapiens
GEHPSPAPPFPSPP